(19) United States
(12) Patent Application Publication
(10) Pub. No.: US 2018/0137247 A1
(43) Pub. Date: May 17, 2018

(54) PREVENTIVE AND PREDICTIVE HEALTH PLATFORM

(71) Applicants: Sigvard Bore, Colorado Springs, CO (US); Sanjay Mohan, Brookfield, WI (US)

(72) Inventors: Sigvard Bore, Colorado Springs, CO (US); Sanjay Mohan, Brookfield, WI (US)

(73) Assignee: healthio Inc., Brookfield, WI (US)

(21) Appl. No.: 15/815,352

(22) Filed: Nov. 16, 2017

Related U.S. Application Data
(60) Provisional application No. 62/422,910, filed on Nov. 16, 2016.

(51) Int. Cl. G06F 19/00; A61B 5/00

(52) U.S. Cl. CPC G06F 19/323; G06Q 50/22; A61B 5/0022; G06F 19/3418

(57) ABSTRACT

A preventive and predictive healthcare platform is provided. This preventive and predictive healthcare platform includes a computer server connected to an electronic communication network. The server has a democratized data store that is owned by the platform user and acts as the single repository of all health information for the platform user (vital signs, medication, wellness profiles, etc.). This data store may be enabled on mobile devices such as smart phones and tablet computers. The server is configured to send and receive healthcare data to healthcare data providers, to process the healthcare data, and to transmit at least some of the healthcare data to one or more users. The preventive and predictive healthcare platform also has an artificial intelligence engine that includes a machine-based learning algorithm which provides a predictive and preventative care model for a platform user.

### CROSS-REFERENCE TO RELATED PATENT APPLICATION

This patent application claims the benefit of U.S. Provisional Patent Application No. 62/422,910, filed Nov. 16, 2016, the entire teachings and disclosure of which are incorporated herein by reference thereto.

### FIELD OF THE INVENTION

This invention generally relates to a participative, preventive and predictive healthcare management platform.

### BACKGROUND OF THE INVENTION

Elder care has long been a problem for many individuals and families in every area of the country. Due to the aging of our population, many families must choose between managed care and in-home care for parent and grandparents. In-home care presents a number of challenges to families that must manage medication and daily medical requirements of elderly family members.

More broadly, managing healthcare for ourselves and our loved ones has become increasingly more challenging for a variety of reasons. The cost of medical treatment and pharmaceuticals put on strain on the finances of many families. Physical separation of family members makes it difficult for patients to find the support needed to effectively care for themselves. Additionally, the rapid advances in medical technologies and the proliferation of choices with respect to providers and types of treatment can be confusing to the average patient, and even more so to elderly patients or patients whose ability to comprehend these choices has been impaired. Moreover, out of the 5000+ waking hours in a year, the average healthcare consumer only spends about 1 or 2 with the healthcare system.

In general, the healthcare system is structured to handle issues or problems after they have occurred rather than analyzing the consumer data to prevent and predict health outcomes. It would therefore be desirable to have a system that provides a means to address some of the aforementioned problems. Such a system would provide patients with a way to more effectively manage their own health issues, and would also provide a way for family members and friends to support the patient in their recovery

The preventive and predictive health platform disclosed herein provides such a system. These and other advantages of the invention, as well as additional inventive features, will be apparent from the description of the invention provided herein.

### BRIEF SUMMARY OF THE INVENTION

In one aspect, embodiments of the invention provide a participative, preventive and predictive healthcare platform. This healthcare platform includes a computer server connected to an electronic communication network. The server sets up a democratized data store that is owned by the platform user and acts as the single repository of all health information for the platform user (vital signs, medication, wellness profiles, etc.). This data store is enabled on mobile devices and that allows the data to move with the platform user—wherever the platform user goes the data goes. The server is also configured to send and receive healthcare data to platform users and their health advocates (friends and family). This data as well as any alert triggered from the data is shared entirely based on consent from the platform user who owns the data.

However, this sharing allows for inter-generational cooperation and increased social interaction to ensure that the platform user is encouraged and motivated to do what is viable for a healthy and independent lifestyle. In certain embodiments of the invention, the preventive and predictive healthcare platform also has an artificial intelligence engine that includes a machine-based learning algorithm which provides a predictive and preventative care model for the platform user. This artificial intelligence engine learns about the platform user, analyzes platform user data automatically and on an on-going basis and generates alerts for medical intervention when required. In a further embodiment of the invention, the server is configured to provide the alert to one or more of the platform user, members of the support network, and a medical care provider.

In particular embodiments, this participative, preventive and predictive platform allows platform users to age at home (rather than managed-care facilities) effectively using the help of friends and family members who have access to data transmissions from the platform.

In another aspect, embodiments of the invention provide a mobile healthcare management platform that includes a computer server connected to an electronic communication network. The server has stored thereon a mobile healthcare application. The server is configured to download the mobile healthcare application to one or more mobile electronic devices of a platform user and of members in a support network of the platform user. The server can access medical records for the platform user. The platform includes one or more sensors for gathering health data from the platform user. The one or more sensors are configured to wirelessly transmit the health data to the mobile healthcare application on at least one of the one or more mobile electronic devices. The server is configured to receive and process the health data from the mobile electronic device of the platform user. The server is further configured to use the health data to update the medical records of the platform user, and configured to determine, based on the medical records and health data of the platform user, a level of risk to the health of the platform user. The server is configured to provide an alert, via the one or more mobile electronic devices, when the level of risk to the health of the platform user is above a predetermined threshold. In a particular embodiment, the server provides a web portal for medical care providers and medical experts to communicate with the platform user.

In an embodiment of the invention, the server includes an artificial intelligence engine that includes a machine-based learning algorithm that provides a predictive and preventative care model for a platform user. In particular embodiments, the artificial intelligence engine is configured to analyze medical histories of multiple third parties and applies lessons learned from that analysis to assess the risk to the health of the platform user. In more specific embodiments, the artificial intelligence engine is configured to evaluate an efficacy of a medical treatment provided to the platform user. Furthermore, the artificial intelligence engine may be configured to generate a recommendation for an individualized medication dose for the platform user.

In certain embodiments, the medical histories of the platform user and of multiple third parties are stored in a database on the server. In more specific embodiments, the medical histories of the platform user and of multiple third parties are anonymized in the database. In a further embodiment, the server includes an encrypted database for storage of personal information of the platform user. More specifically, the encrypted database may be a SOX2/3 secured cloud database.

In some embodiments, the artificial intelligence engine is configured to determine the level of risk based on an analysis of the medical records of the platform user and of multiple third parties. More specifically, the artificial intelligence engine may be configured to automatically update the level of risk as the medical records are updated. In certain embodiments, the artificial intelligence engine is configured to automatically determine the predetermined threshold based on an analysis of the medical records of the platform user and of multiple third parties. Furthermore, the artificial intelligence engine may also be configured to automatically update the predetermined threshold as the medical records are updated.

In a particular embodiment of the invention, the artificial intelligence engine is configured to automatically provide educational messages and recommendations to platform users based on the individual needs of the platform user. In another embodiment, the artificial intelligence engine is configured to automatically provide real-time on-demand video medical consultation for the platform user. In a specific embodiment, the artificial intelligence engine comprises at least one of a data collection, capturing, and completion layer, a data storage and retrieval layer, a data aggregation and enrichment layer, a data mining and classification layer, and an information delivery layer.

In a particular embodiment of the invention, the artificial intelligence engine is configured to automatically determine an acceptable range of results for the health data gathered by the one or more sensors, and wherein the server transmits an alert to the mobile electronic devices of the platform user and support network when the health data is outside of the acceptable range. In more specific embodiments, the artificial intelligence engine is configured to generate a predictive outcome for a specified course of treatment undertaken by the platform. Furthermore, the artificial intelligence engine may be configured to facilitate medical specialist intervention based on the level of risk and an analysis of the medical records of the platform user and of multiple third parties.

In a further embodiment of the mobile healthcare management platform, the one or more sensors comprise one of a blood pressure monitor, an oxygen saturation pulse oximeter, a spirometer, a thermometer, an activity tracker, a weight scale, an electrocardiograph (ECG), and a glucometer. In a particular embodiment, the one or more sensors communicate wirelessly with the one or more mobile electronic devices using the Bluetooth Low Energy protocol. In another embodiment of the invention, the one or more mobile electronic devices comprise one of a smartphone, a smartwatch, a tablet computer, a notebook computer, a laptop computer, and a personal digital assistant.

In certain embodiments, the mobile healthcare application is configured to accept medical data input manually into the one or more mobile electronic devices. Additionally, the mobile healthcare application may be configured to transmit the manually input medical data to the server which updates the medical records of the platform user with the manually input medical data.

In a particular embodiment, the server is configured to aggregate and store medical records from one or more of healthcare provider networks, medical testing labs, insurance companies, healthcare affiliates, pharmacies, and pharmacy benefit managers.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention.

FIG. 1 is a schematic representation of the mobile healthcare management platform, according to an embodiment of the invention;

FIG. 2 is an illustration of various exemplary medical sensors, in accordance with an embodiment of the invention;

FIG. 3 is a schematic representation of the mobile healthcare management platform showing how information flows in the platform, according to an embodiment of the invention;

FIG. 4 is a diagram showing the development process for the artificial intelligence engine, according to an embodiment of the invention; and

FIG. 5 is a diagram showing data capture and information delivery with respect to the artificial intelligence engine, according to an embodiment of the invention.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

Embodiments of the present invention include a mobile, preventive and predictive healthcare platform that includes a computer server connected to a communication network, such as the internet. In particular embodiments of the invention, the server is configured to receive and process healthcare information from healthcare provider networks, medical diagnostic laboratories, health insurance companies, pharmacy benefit managers, and pharmacies. The server may be further configured to process the volumes of healthcare data and provide the relevant data to the platform user. The healthcare data may be transmitted over the communication network from the server to one or more mobile electronic devices, such as a tablet, smartphone, smartwatch, laptop computer, notebook computer, personal digital assistant, or to one or more personal computers belonging the platform user and members of the support network for the platform user. However, healthcare data from one or more sensors used by the platform user, or from information manually entered into the mobile healthcare application, may flow from the mobile electronic devices or personal computers to the server.

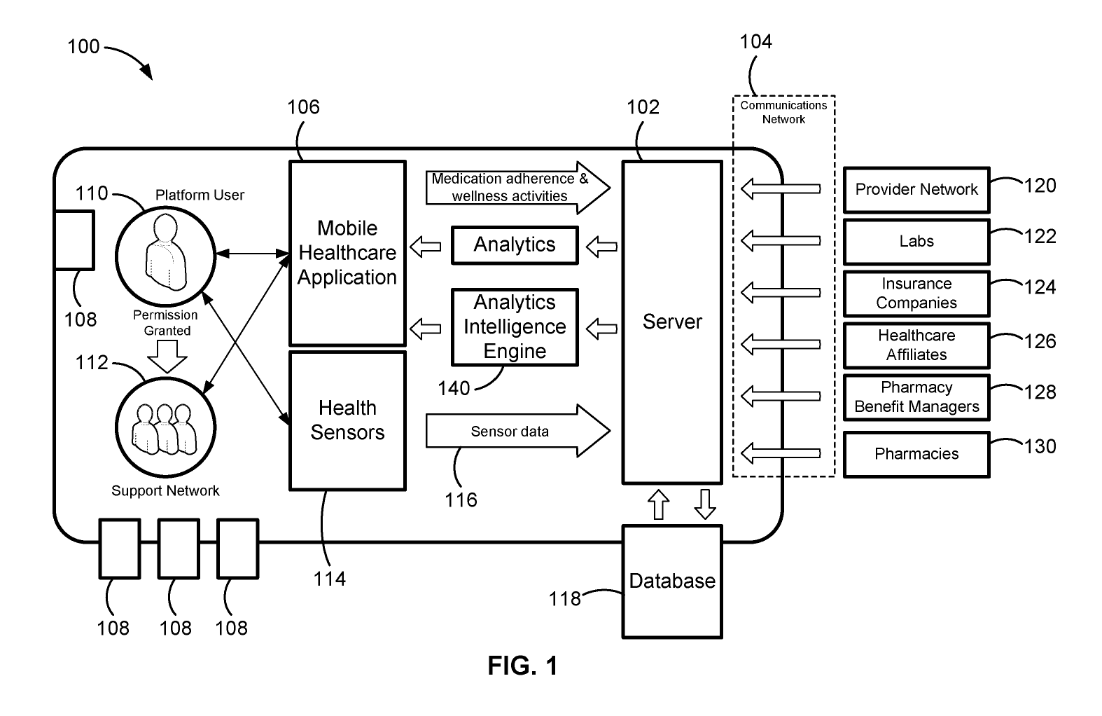

FIG. 1 shows a schematic representation of the mobile healthcare management platform 100 which includes a computer server 102 connected to an electronic communication network 104, such as the internet. As shown in FIG. 1, the server 102 communicates, via the electronic communication network 104, with healthcare provider networks 120, medical diagnostic laboratories 122, health insurance companies 124, healthcare affiliates 126 (e.g., the American Heart Association (AHA), the American Red Cross, the American Association of Retired Persons (AARP), the American Diabetes Association (ADA), the American Cancer Society, etc.) pharmacy benefit managers 128, and pharmacies 130. The server 102 has stored thereon a mobile healthcare application 106. The server 102 is configured to download the mobile healthcare application 106 to one or more mobile electronic devices 108 of a platform user 110 and of members in a support network 112 of the platform user 110. The server 102 is configured to access medical and personal records for the platform user 110. Those records include, but are not limited to, radiological records, medical prescription records, genomic data, lab test results, insurance claim history, occupational history, family history, etc. The server 102 is configured to use the aforementioned medical and personal data to develop treatment recommendations, reminders, automated alerts, etc.

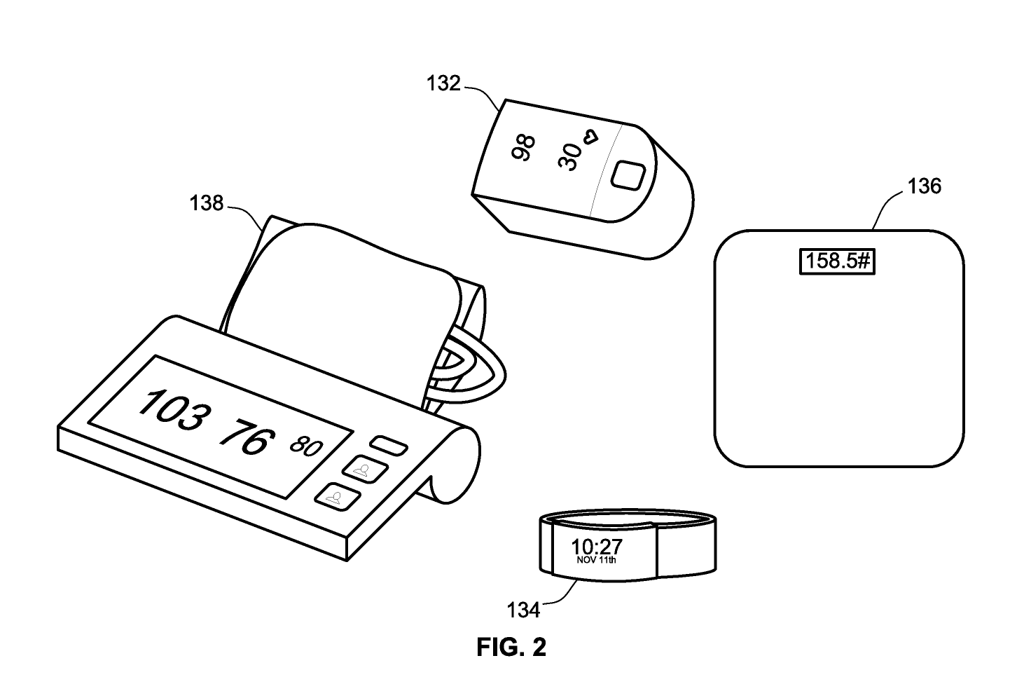

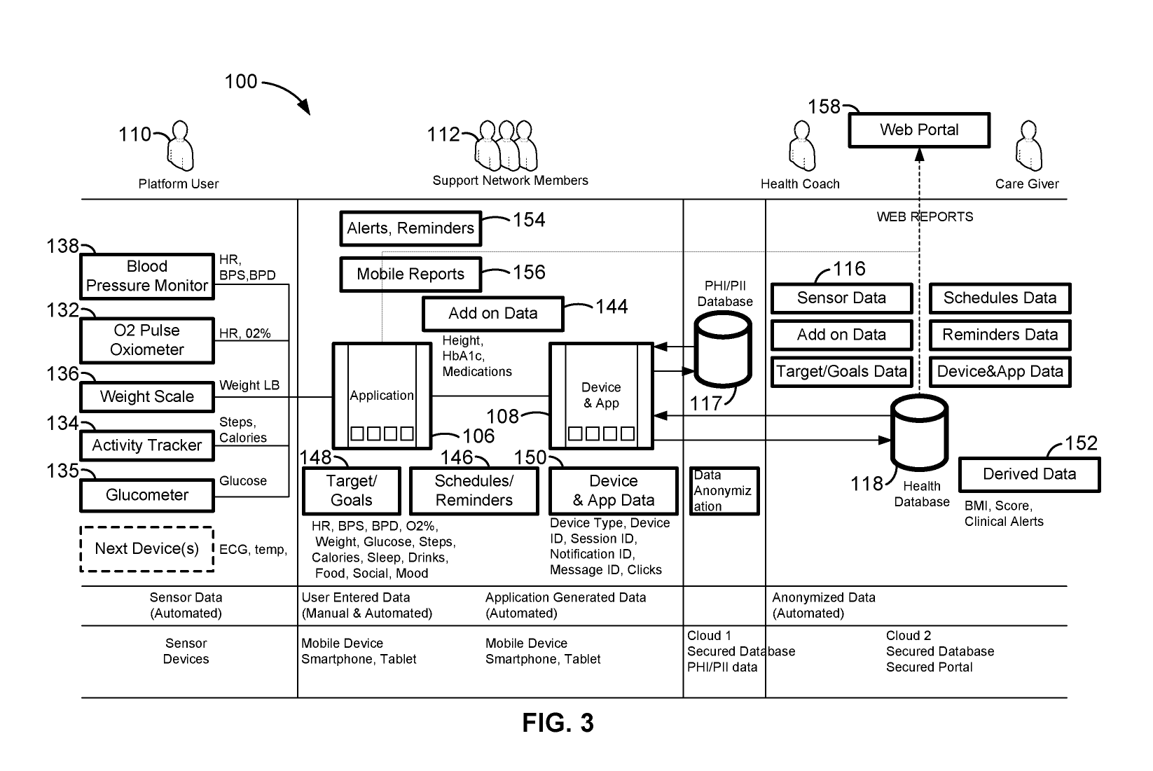

The platform 100 also includes one or more sensors 114 for gathering health data 116 from the platform user 110. FIG. 2 illustrates various sensors 114 that can be used in embodiments of the mobile healthcare management platform 100 described herein. The one or more sensors 114 may include, but are not limited to, an oxygen saturation pulse oximeter 132, a spirometer, a thermometer, an activity tracker 134, a weight scale 136, a blood pressure monitor 138, an electrocardiograph (ECG), and a glucometer 135 (in FIG. 3). In particular embodiments, the one or more sensors 114 are configured to communicate wirelessly with the mobile healthcare application 106 in the one or more mobile electronic devices 108 using the Bluetooth Low Energy (BLE) protocol.

The one or more sensors 114 are configured to wirelessly transmit the health data 116 to the mobile healthcare application 106 on at least one of the one or more mobile electronic devices 108. The one or more mobile electronic devices 108 may include, but are not limited to, a smartphone, a smartwatch, a tablet computer, a notebook computer, a laptop computer, or a personal digital assistant. The data transmitted by the sensors 114 provide a continuous stream of medical data which can be monitored to see how the data changes over time. This continuous stream of medical data allows for significant improvements in diagnoses as compared with conventional medical care where physicians typically make diagnoses based on data collected during a single visit. Because a person's condition can vary with the time of day, time of month, and even time of year, basing a diagnosis on data collected at one point in time carries definite disadvantages. Diagnostic accuracy can be greatly improved when the medical data available to the physician covers the weeks or months leading up to the diagnosis.

FIG. 3 is a schematic illustration of the mobile healthcare management platform 100 showing how information flows in the platform 100, according to an embodiment of the invention. In the example shown in FIG. 3, sensor data is transmitted automatically from the one or more sensors 114, via the Bluetooth Low Energy protocol, to the mobile healthcare application 106 in one or more mobile electronic devices 108. Whenever the sensors 114 acquire new information, that information is sent wirelessly to the mobile healthcare application 106 in the mobile electronic device 108 of the platform user 110. This information is then sent to the server 102 for storage in the health database 118. As will be shown below, in practice, the platform 100 may actually use multiple databases to store the various types of data, rules, guidelines, and algorithms employed to run the platform 100.

In the embodiment of FIG. 3, after each measurement, the blood pressure monitor 138 wirelessly transmits heart rate data, along with systolic and diastolic blood pressure data to the mobile healthcare application 106 in the mobile electronic device 108 of the platform user 110. Similarly, after each measurement, the oxygen saturation pulse oximeter 132 wirelessly transmits heart rate data, along with oxygen level data. The glucometer 135 wirelessly transmits glucose levels, the activity tracker 134 transmits the number of steps taken and calories burned, while the scale 136 transmits the platform user's weight. Additionally, the mobile healthcare application 106 accepts manually-input data, or add-on data 144, such as prescribed medications, medical conditions, height, HbA1c levels, schedules 146, activities, targets/goals 148, etc.

In embodiments of the invention, the mobile healthcare application 106 is configured to generate device and application data 150 used for two-way communication between the mobile electronic device 108 and the server 102. In the embodiment of FIG. 3, the mobile healthcare application 106 generates a device type, and device ID for the mobile electronic device 108 hosting the mobile healthcare application 106. The mobile healthcare application 106 is also configured to generate a message ID, a session ID, and a notification ID.

Health data flowing from the mobile electronic device 108, to the encrypted, HIPAA-compliant database 117 and to the health database 118 of server 102, is anonymized. As shown in FIG. 3, in addition to health data 116 from sensors 114, the health database 118 holds the add-on data 144, the schedules 146, activities, targets/goals 148, the device and application data 150. Using this stored data, the server 102 is configured to generate derived data 152 which includes, but is not limited to, automated alerts and reminders 154, mobile reports 156, other parameters like body/mass index, or calculated health risk scores or ratings which represent a level of risk to the health of the platform user 110. As explained below, the server 102 could be configured to determine a threshold for the health risk score, such that a score above the threshold indicates an unacceptably high risk to the health of the platform user 110. The server 102 could be configured to automatically calculate the health risk score and the threshold for the health risk score. Alternatively the health risk score and threshold could be determined with the input of experts such as medical care providers, health coaches, pharmaceutical experts, etc. who can access the server 102 and provide input through a web portal 158.

The server 102 is configured to receive and process the health data 116 from the mobile electronic device 108 of the platform user 110. The server 102 is further configured to use the health data 116 to update the medical records of the platform user 110, and configured to determine, based on the medical records and health data 116 of the platform user 110, a level of risk to the health of the platform user 110. The server 102 is configured to automatically provide an alert 154, via the one or more mobile electronic devices 108, when the level of risk to the health of the platform user 110 is above a predetermined threshold. In determining the level of risk to the platform user 110, the server may rely on data provided by medical care providers or other medical and pharmaceutical experts.

In a particular embodiment, the application architecture integrates a number of third-party leading industry system solutions to send user verification and authentication codes, push notifications or in-application messages, check and auto-fill medications. The mobile healthcare application 106 is configured to generate clinical alerts 154 based on condition protocols and clinical rules when measurements are consistently outside of predetermined or accepted thresholds.

The platform's data architecture is designed to collect and to aggregate very large sensor and non-sensor data sets and to integrate seamlessly with other clinical data sources, enabling data mining and artificial intelligence algorithms such as profile similarity search, treatment efficiency, and medication dose personalization. As the number of devices supporting Bluetooth Low-Energy (BLE) protocols continues to grow, the flexible and modular architecture of the platform 100 is designed to allow for the quick integration of additional devices such as electrocardiographs (ECG), thermometers, spirometers so platform users 110 can completely monitor their health at home.

The mobile healthcare application 106 is designed for users, e.g., support network members 112, and health care organizations to monitor the health status of platform users 110 with various health conditions outside of provider settings and to increase compliance and “just in time” interventions by enabling data sharing and clinical alerts 154 with support network members 112, family members, or primary care givers. Embodiments of the mobile healthcare application 106 are configured to allow the platform user 110 to add members to, or remove members from, the support network 112.

The mobile healthcare management platform 100 provides a system in which frequent and real time readings from Bluetooth-enabled medical sensors 114 and fitness data from activity trackers 134 coupled with behavioral data entered by platform user 110 or support network members 112, compliance and other real-time measurement data for the platform user 110, can be organized and shared among various individuals and professionals who are involved in caring for the platform user 110.

In addition to data sharing in a secured, private cloud environment, the mobile healthcare platform 100 integrates capabilities for people in the various support network member 112 and caregiver groups to communicate with each other in real-time to collaborate on the caring of the platform user 110. The platform user 110 is provided with a complete set of medical devices, or sensors 114, that are easy to use, and a mobile healthcare application 106 that, in some embodiments, is downloadable from popular third-party portals such as, for example, Apple's App Store, the AmazonAppstore, or Google's Play Store.

The mobile healthcare application 106 enables platform users 110 to take manual or automated measurements and to record personal health data points into a secure and scalable cloud database 118. The support network members 112 are provided with the same mobile healthcare application 106 and access to the individual data, as enabled by the platform user 110. In addition, the support network members 112 can initiate and coordinate the devices procurement and the application setup for their loved ones. To minimize security risks, any sensitive personal data (i.e., protected health information (PHI) or personally identifiable information (PII)) is separated and encrypted in a HIPAA-compliant, isolated database 117 (see FIG. 3), while the shared health data is stored in an accessible portion of the health database 118. In more specific embodiments, the HIPAA-compliant database 117 could be a SOX2/3 secured cloud that stores the health records collected from devices, and which communicates the PHI/PII data via secured token protocols with the health database 118.

In certain embodiments, the mobile healthcare application 106 is configured to accept medical information input manually into the one or more mobile electronic devices 108. This medical information may be input by the platform user 110 or by a member of the support network 112. Additionally, the mobile healthcare application 106 may be configured to transmit the manually-input medical information to the server 102 which would update the medical records of the platform user 110 with the manually input medical information. For example, the platform user 110 could enter information into the mobile healthcare application 106 related to the platform user's adherence to prescribed or over-the-counter medication, or information related to diet and exercise and other wellness activities.

The mobile healthcare application 106 is configured to prompt the platform user to manually input relevant information, and transmit the information to the server 102 where the data is aggregated with other information on the platform user 110. Three key elements used by the platform 100 to determine the health of the platform user 110 are vital signs, medication, and general wellness. The sensors 114 generally provide medical data for the vital signs, while medical records from pharmacies, health care providers, hospitals, etc. may provide data on medications taken by the platform user 110. Key factors related to general wellness include mood, pain, diet, sleep, digestion, exercise, etc. To adequately gauge these factors, the mobile healthcare application 106 requests input from the platform user 110. As needed, the server 102 sends questions, via the mobile healthcare application 106, regarding the platform user's mood, pain, diet, sleep, digestion, exercise, etc. The information provided by the platform user 110 is analyzed in concert with other data collected on the platform 100, and used to generate recommendations and aggregated with other medical data to determine a health risk score and threshold score for the platform user 110.

Platform users 110 of any age can utilize this platform 100 for the ongoing management of medical issues and chronic conditions, and, using the artificial intelligence engine 140, can use the platform 100 for predicting ailments or health risks that might be eliminated or reduced by immediate medical intervention.

The healthcare data 116, which is updated in real time, can be accessed at any time by family members and support network members 112 for a platform user 110. The access to healthcare data 116 allows for family and support network members 112 to plan and coordinate care and assistance for the platform user 110.

In a particular embodiment of the invention, the preventive and predictive healthcare platform 100 also includes an artificial intelligence (AI) engine 140, which may be located on the computer server, designed to provide predictive medical analysis. The artificial intelligence engine 140 includes a machine-based learning algorithm that provides a predictive and preventative care model for the platform user 110, which can be implemented by the family members and friends (i.e., support network members 112) connected to the preventive and predictive healthcare platform 100.

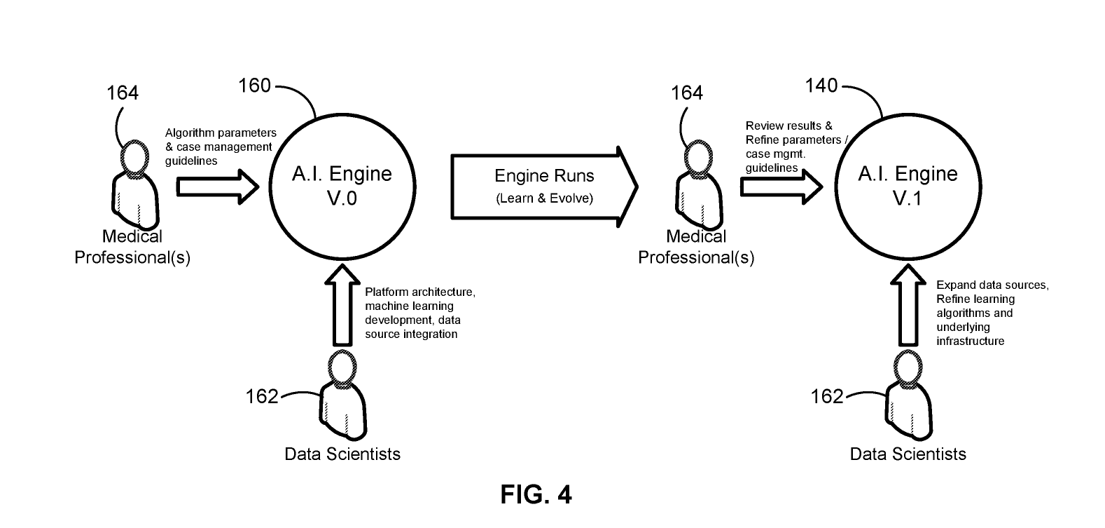

FIG. 4 is a diagram showing the development process for the artificial intelligence engine 140, according to an embodiment of the invention. In its initial stages, the new artificial intelligence engine 160 includes platform architecture, along with programming to facilitate machine learning development and data source integration provided primarily by data scientists 162. This is combined with algorithm parameters and case managements guidelines provided primarily by medical experts 164. In operation the new artificial intelligence engine 160 learns from interactions with platform users 110 and support network members 112. As the new artificial intelligence engine 160 learns and evolves, the medical experts 164 are able to review the results of the interactions and refine the algorithm parameters and case managements guidelines, while the data scientists 162 expand data sources, and refine the machine learning algorithms and underlying infrastructure to produce the fully developed artificial intelligence engine 140.

This artificial intelligence engine 140 learns about the platform user 110, analyzes the health data 116 of the platform user 110 automatically and on an on-going basis, and generates a reminder 154 for the platform user 110, or alerts 154 for medical intervention when required. These alerts 154 are transmitted from the server 102 to the mobile electronic devices 108 of the platform user 110 and the support network members 112, and possibly to a medical care provider, to prompt some action or response in response to the alert 154.

In an embodiment of the invention, the artificial intelligence engine 140 is configured to provide at least ten types of actionable information to platform users 110 and support network members 112. While it is envisioned that the functionality of the artificial intelligence engine 140 will increase over time, platform users 110 and support network members 112 will receive at least the following communications.

### 1. Generating Alerts of Condition Worsening

In real-time situations, the artificial intelligence engine 140 can warn of changes in a platform user's condition, based on repeated patterns of measurements.

### 2. Generating Reminders for Scripts or Visits

The artificial intelligence engine 140 may scan laboratory test results or drug orders and send reminders or warnings through an e-mail system or text messaging.

### 3. Generating Alerts of Non-Compliance to Treatment

The artificial intelligence engine 140 can detect situations in which the platform user 110 is not complaint to standards of care, and subsequently alert support network members 112 for outreach purposes.

### 4. Providing Diagnostic Assistance

When a platform user's case is complex and rare, the artificial intelligence engine 140 can “suggest” the “likely” diagnosis based on platform user data, and advise the platform user 110 to seek immediate care.

### 5. Identification of Best Quality Providers to Treat Condition

The artificial intelligence engine 140 can look for best quality rated providers in the geographical area for various medical specialties and for a variety of treatments and make recommendations based on travel distance, volume, and published quality metrics

### 6. Identifying Gaps in Care or Therapy Planning

The artificial intelligence engine 140 can look for inconsistencies, errors and omissions in an existing treatment plan, or can be used to formulate a treatment plan based upon a platform user's specific condition and accepted treatment guidelines.

### 7. Clinical Guidelines Information Retrieval

The artificial intelligence engine 140 can search for (e.g., the internet) and retrieve information that is considered relevant to a particular problem. The artificial intelligence engine 140 contains knowledge about the platform user's preferences and needs, and may also have medical knowledge to be able to assess the importance and utility of what it finds.

### 8. Image Recognition and Interpretation

In addition to ECG, the artificial intelligence engine 140 can flag potentially abnormal images for detailed human attention based on CT, MRI image analysis.

### 9. Identifying Drug to Drug Adverse Effects

In situations where users are taking a series of medications at the same time, the artificial intelligence engine 140 can warn of potential drug to drug negative interactions

### 10. Personalized Medicine Parameters:

The artificial intelligence engine 140 can build the platform user's unique profile based on specific characteristics and compare to similar populations, detecting best treatments that provide most optimal clinical and economical outcomes, such as:
i) Appropriate Medication Doses that Provides Best Outcomes for the Platform User 110
“Metformin 250 Mg is optimal dose for this medication to decrease A1C ratio by 1 point for this kind of platform user 110”

### ii) Best Combination of Treatments Providing Best Outcomes for the Platform User 110

“Medication only treatment without surgery worked for 90% of the similar patients or alternative medicine therapy heal these cases in 90% of times
iii) Correlations Between Specific Lifestyle Characteristics and Health Status
“30 minutes exercise per day and 9000 steps is the optimal amount of physical activity for this kind of platform user 110.

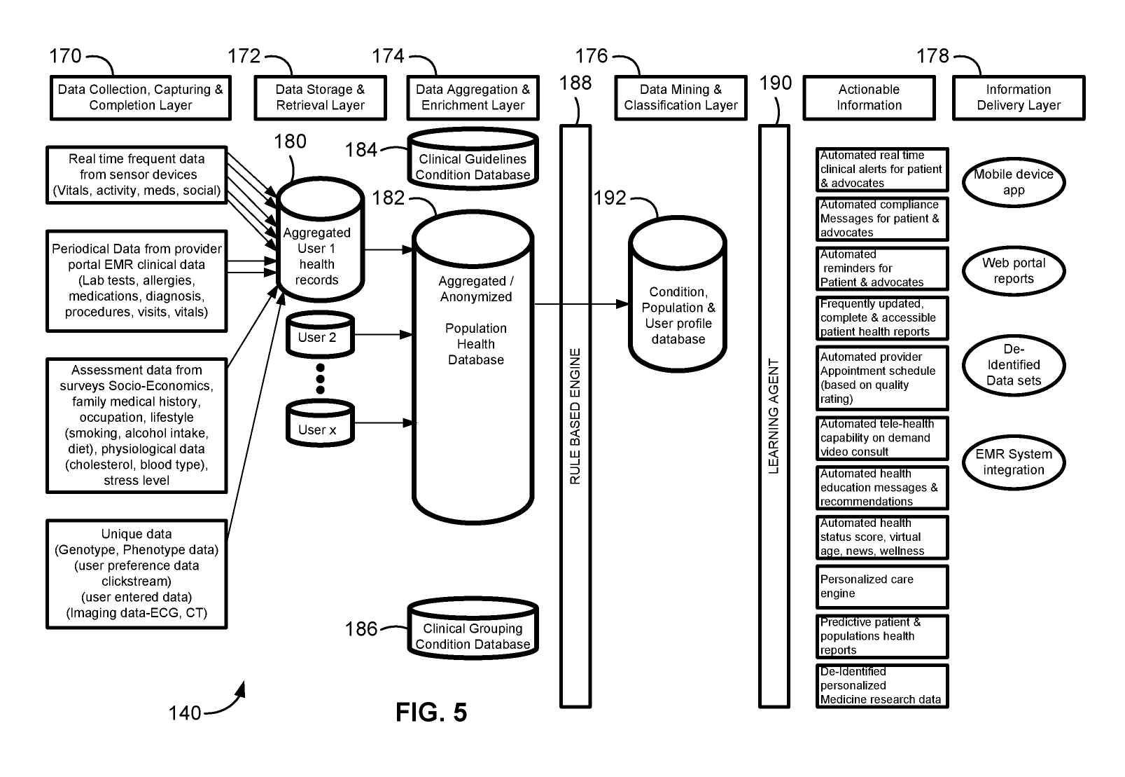

FIG. 5 is a diagram showing data capture and information delivery with respect to the artificial intelligence engine 140, according to an embodiment of the invention. The artificial intelligence engine 140 of FIG. 5 includes a data collection, capturing, and completion layer 170, a data storage and retrieval layer 172, a data aggregation and enrichment layer 174, a data mining and classification layer 176, and an information delivery layer 178.

The data collection, capturing, and completion layer 170 collects data from a variety of sources such as the sensors worn by the platform user 110, medical laboratory test results, medical periodicals, manually-input data from the platform user 110, genomic data, medical research results, government regulations, etc. The data storage and retrieval layer 172 includes storage of the aggregated personal and health records 180 of the platform user 110. Further, the database also includes health records for multiple third party platform users. In the data aggregation and enrichment layer 174, the health records of the platform user 110 along with those of multiple third party platform users are aggregated and anonymized in a population health database 182. The data aggregation and enrichment layer 174 also includes a clinical guidelines condition database 184 and clinical grouping condition database 186, where these two databases contain rules and guidelines, provided by the medical experts 164, by which the artificial intelligence engine 140 operates.

A rule-based engine 188 and a learning agent 190 apply the rules and algorithms (provided by the data scientists 162 and medical experts 164 to the aggregated and anonymized data in the population health database 182 in order to generate useful information for the platform users 110 and support network members 112 (such as the 10 communication examples recited above). The useful information generated by the rule-based engine 188 and a learning agent 190 is stored in a condition, population and user profile database 192. This useful information is delivered to platform users 110 and support network members 112 via the information delivery layer 178 through the mobile healthcare application 106, reports sent to the web portal 158, de-identified data sets, EMR system integration, etc.

In particular embodiments, the predictive and preventative care model is facilitated by the artificial intelligence engine 140 which is configured to analyze medical histories of multiple third parties and apply lessons learned from that analysis to assess the various risks to the health of the platform user 110. In more specific embodiments, the artificial intelligence engine 140 is configured to evaluate the efficacy and efficiency of the medical treatment options available to the platform user 110. Furthermore, the artificial intelligence engine 140 may be configured to generate specific recommendations for an individualized medication dose for the platform user 110.

In certain embodiments, the medical histories of the platform user and of multiple third parties are stored in the database 118 on the server 102. In some embodiments, the artificial intelligence engine 140 is configured to determine the level of risk based on an analysis of the medical records of the platform user 110 and of multiple third parties. More specifically, the artificial intelligence engine 140 may be configured to automatically update the level of risk as the medical records are updated. In certain embodiments, the artificial intelligence engine 140 is configured to automatically determine the predetermined threshold based on an analysis of the medical records of the platform user 110 and of multiple third parties. Furthermore, the artificial intelligence engine 140 may also be configured to automatically update the predetermined threshold as the medical records are updated.

In a particular embodiment of the invention, the artificial intelligence engine 140 is configured to determine an acceptable range of results for the health data 116 gathered by the one or more sensors 114, and wherein the server 102 transmits an alert 154 to the mobile electronic devices 108 of the platform user 110 and support network members 112 when the health data 116 is outside of the acceptable range. In more specific embodiments, the artificial intelligence engine 140 is configured to generate a predictive outcome for a specified course of treatment undertaken by the platform user 110.

In more specific embodiments of the invention, the artificial intelligence engine 140 is configured to provide automated on demand video medical consultations to address medical concerns and to answer medical questions from the platform user 110 and support network members 112. Thus, it is envisioned that the artificial intelligence engine 140 will have access to an extensive medical library in order to address a variety of medical issues from system users in real-time.

Additionally, the artificial intelligence engine 140 is configured to send out health education messages and recommendations to platform users 110 based on the individual needs of the platform user 110. These education messages and recommendations may be released in response to new medical research results or new data from government or private medical agencies. It is also envisioned that medical care providers, health coaches, and other medical experts will be able to use the web portal 158 to send health education messages and recommendations to specific platform users 110.

In a further embodiment, the artificial intelligence engine 140 generates education messages directed to support network members 112. For example, if the artificial intelligence engine 140 determines that the most relevant factor influencing the health of the platform user 110 is communication with and the involvement of support network members 112, the artificial intelligence engine 140 can transmit, via the server 102, recommendations to the support network members 112 with advice for assisting the platform user 110.

In one example of how the artificial intelligence engine 140 may function to help the platform user 110 with hypertension. As referenced above, the artificial intelligence engine 140 has the advantage provided by a continuous stream of blood pressure and heart rate data from the Bluetooth-enabled blood pressure monitor 138. With conventional medical care, the physician must often make decisions based solely on the blood pressure readings taken during infrequent patient visits. Because blood pressure readings may vary during the day, week, or month, the data collected by the blood pressure monitor 138 gives the physician a more complete picture of the platform user's health.

Furthermore, the artificial intelligence engine 140 is able to analyze how blood pressure responds to medication immediately and over time, and can also correlate blood pressure changes to actions taken or not taken by support team members 112. The artificial intelligence engine 140 can also look at how blood pressure rises and falls to determine possible causes. In addition to analyzing blood pressure changes in the platform user 110, the artificial intelligence engine 140 can also look at hypertension in other populations to determine the common denominators associated with positive outcomes and common factors associated with negative outcomes. Using knowledge gained from analysis of other populations allows the artificial intelligence engine 140 to make additional recommendations or warnings to improve results for the platform user 110.

As described hereinabove, the present invention is a mobile healthcare management platform 100 with a server 102 that provides a democratized data store owned by the platform user 110 and which acts as the single repository for all of the platform user's health information (e.g., vital signs, medication, wellness profiles, etc.). This data store may be enabled on one or more mobile electronic devices 108, such as smartphones, smartwatches, tablet computers, and larger personal computers. This type of two-way communication allows the health data 116 to move with the platform user 110—i.e., wherever the platform user 110 goes, the data 116 goes. The server 102 is also configured to send and receive healthcare data 116 to platform users 110 and their support network members 112.

This data 116, as well as any alert 154 triggered from the data 116, is shared entirely based on consent from the platform user 110 who owns the data. However, this sharing allows for inter-generational cooperation and increased social interaction to ensure that the platform user 110 is encouraged and motivated to do what is viable for a healthy and independent lifestyle. Participative healthcare platforms, such as the preventive and predictive platform 100 disclosed herein, allow platform users 110 to age at home, rather than in managed-care facilities, effectively using the help of friends and family members (support network members 112) who have access to data transmissions from the platform 100.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A mobile healthcare management platform comprising:
a computer server connected to an electronic communication network, the server having stored thereon a mobile healthcare application, the server being configured to download the mobile healthcare application to one or more mobile electronic devices of a platform user and of members in a support network for the platform user, wherein the server can access medical records for the platform user;
one or more sensors for gathering health data from the platform user, the one or more sensors configured to wirelessly transmit the health data to the mobile healthcare application on at least one of the one or more mobile electronic devices;
wherein the server is configured to receive and process the health data from the mobile electronic device of the platform user, the server being further configured to use the health data to update the medical records of the platform user, and configured to determine, based on the medical records and health data of the platform user, a level of risk to the health of the platform user;
wherein the server is configured to automatically provide an alert, via the one or more mobile electronic devices, when the level of risk to the health of the platform user is above a predetermined threshold.

2. The mobile healthcare management platform of claim 1, wherein the server includes an artificial intelligence engine that includes a machine-based learning algorithm that provides a predictive and preventative care model individualized for the platform user.

3. The mobile healthcare management platform of claim 2, wherein the artificial intelligence engine is configured to analyze medical histories of multiple third parties and applies lessons learned from that analysis to assess the risk to the health of the platform user.

4. The mobile healthcare management platform of claim 3, wherein the artificial intelligence engine is configured to evaluate an efficacy of a medical treatment provided to the platform user.

5. The mobile healthcare management platform of claim 3, wherein the artificial intelligence engine is configured to generate a recommendation for an individualized medication dose for the platform user.

6. The mobile healthcare management platform of claim 3, wherein the medical histories of the platform user and of multiple third parties are stored in a database on the server.

7. The mobile healthcare management platform of claim 6, wherein the medical histories of the platform user and of multiple third parties are anonymized in the database.

8. The mobile healthcare management platform of claim 3, wherein the artificial intelligence engine is configured to determine the level of risk based on an analysis of the medical records of the platform user and of multiple third parties.

9. The mobile healthcare management platform of claim 8, wherein the artificial intelligence engine is configured to update the level of risk as the medical records are updated.

10. The mobile healthcare management platform of claim 8, wherein the artificial intelligence engine is configured to facilitate medical specialist intervention based on the level of risk and an analysis of the medical records of the platform user and of multiple third parties.

11. The mobile healthcare management platform of claim 8, wherein the artificial intelligence engine is configured to automatically determine the predetermined threshold based on an analysis of the medical records of the platform user and of multiple third parties.

12. The mobile healthcare management platform of claim 11, wherein the artificial intelligence engine is configured to update the predetermined threshold as the medical records are updated.

13. The mobile healthcare management platform of claim 2, wherein the artificial intelligence engine is configured to determine an acceptable range of results for the health data gathered by the one or more sensors, and wherein the server automatically transmits an alert to the mobile electronic devices of the platform user and support network when the health data is outside of the acceptable range.

14. The mobile healthcare management platform of claim 2, wherein the artificial intelligence engine is configured to generate a predictive outcome for a specified course of treatment undertaken by the platform.

15. The mobile healthcare management platform of claim 2, wherein the artificial intelligence engine is configured to automatically provide educational messages and recommendations to platform users based on the individual needs of the platform user.

16. The mobile healthcare management platform of claim 2, wherein the artificial intelligence engine is configured to automatically provide real-time on-demand video medical consultation for the platform user.

17. The mobile healthcare management platform of claim 2, wherein the artificial intelligence engine comprises at least one of a data collection, capturing, and completion layer, a data storage and retrieval layer, a data aggregation and enrichment layer, a data mining and classification layer, and an information delivery layer.

18. The mobile healthcare management platform of claim 1, wherein the one or more sensors comprise one of a blood pressure monitor, an oxygen saturation pulse oximeter, a spirometer, a thermometer, an activity tracker, a weight scale, an electrocardiograph (ECG), and a glucometer.

19. The mobile healthcare management platform of claim 1, wherein the one or more sensors communicate wirelessly with the one or more mobile electronic devices using the Bluetooth Low Energy protocol.

20. The mobile healthcare management platform of claim 1, wherein the mobile healthcare application is configured to accept medical data input manually into the one or more mobile electronic devices.

21. The mobile healthcare management platform of claim 20, wherein the mobile healthcare application is configured to transmit the manually input medical data to the server which updates the medical records of the platform user with the manually input medical data.

22. The mobile healthcare management platform of claim 1, wherein the server is configured to aggregate and store medical records from one or more of healthcare provider networks, medical testing labs, insurance companies, healthcare affiliates, pharmacies, and pharmacy benefit managers.

23. The mobile healthcare management platform of claim 1, wherein the one or more mobile electronic devices comprise one of a smartphone, a smartwatch, a tablet computer, a notebook computer, a laptop computer, and a personal digital assistant.

24. The mobile healthcare management platform of claim 1, wherein the server is configured to automatically provide the alert to one or more of the platform user, members of the support network, and a medical care provider.

25. The mobile healthcare management platform of claim 1, wherein the mobile healthcare application is configured to allow the platform user to add or remove members to the support network.

26. The mobile healthcare management platform of claim 1, wherein the server provides a web portal for medical care providers and medical experts to communicate with the platform user.

27. The mobile healthcare management platform of claim 1, wherein the server includes an encrypted database for storage of personal information of the platform user.

28. The mobile healthcare management platform of claim 27, wherein the encrypted database is a SOX2/3 secured cloud database.
